(19) United States
(12) Patent Application Publication
(10) Pub. No.: US 2013/0078207 A1
(43) Pub. Date: Mar. 28, 2013

(54) Artificial Nail Composition, Nail Covering Agent, And Method For Forming Cover Film

(71) Applicant: Masahiko SANBONMATSU, Osaka-shi (JP)

(72) Inventor: Masahiko SANBONMATSU, Osaka-shi (JP)

(73) Assignee: MATSUKAZE CO., LTD., Osaka (JP)

(21) Appl. No.: 13/617,765

(22) Filed: Sep. 14, 2012

(30) Foreign Application Priority Data
Sep. 27, 2011 (JP) 2011-211092
Nov. 9, 2011 (JP) 2011-006613
Feb. 9, 2012 (JP) 2012-026278

(51) Int. Cl. A61K 8/72; A61Q 3/02; A45D 29/12; A61K 8/73

(52) U.S. Cl. 424/61; 401/126; 401/143

(57) ABSTRACT

A novel artificial nail composition (gel nail composition) is provided. The artificial nail composition is curable with ultraviolet rays to form an artificial nail, and includes an ultraviolet-curable resin and pearl powder dispersed in the ultraviolet-curable resin.

### BACKGROUND OF THE INVENTION

1. Field of the Invention

The present invention relates to an artificial nail composition, a nail covering agent, and a method for forming a cover film, and more specifically to an artificial nail composition (gel nail composition) which is cured when being irradiated with ultraviolet rays to form an artificial nail (so-called gel nail).

The present application claims the benefit of priority based upon Japanese Patent Application No. 2011-211092 filed on Sep. 27, 2011, Japanese Utility Model Application No. 2011-6613 filed on Nov. 9, 2011, and Japanese Patent Application No. 2012-26278 filed on Feb. 9, 2012. The entirety of the contents of these applications is incorporated herein by reference.

2. Description of the Related Art

Recently, nail art of designing finger nails of hands or feet becomes increasingly popular, and various technologies for forming an artificial nail (false nail) of a synthetic resin on the nail (human nail) have been proposed. Among these technologies, an artificial nail (gel nail) formed by applying a gel-like artificial nail composition (gel nail composition) containing a urethane-based resin, an acrylic resin or the like and a photopolymerizable monomer on a nail and then irradiating and thus curing the artificial nail composition is attracting an attention today for the reason that, for example, the finish is clear (see, for example, Japanese Patent Laid-Open Publication No. 2009-126833).

Another reason that a gel nail is popular is that the gel nail has a high adhesiveness with the human nail and thus lasts longer as compared with manicure or the like. Namely, manicure comes off within several days in everyday life, whereas gel nails mostly last for several weeks and thus are popular.

### SUMMARY OF THE INVENTION

Some gel nails have a pearl color. However, in actuality, the gel nail composition does not contain pearl. What is available today is merely a so-called pseudo-pearl; namely, a pearl-like color is produced by a pigment. The present inventor found the following: when pearl powder or mother-of-pearl powder is incorporated into the gel nail composition, the fact that pearl or mother-of-pearl is incorporated can be exhibited; and functionality can be improved by incorporating such powder. As a result, the present inventor has achieved the present invention.

The present invention made in light of such a situation has a main object of providing a novel artificial nail composition (gel nail composition).

An artificial nail composition according to the present invention is an artificial nail composition for forming an artificial nail, the artificial nail composition being curable with ultraviolet rays. The artificial nail composition comprises an ultraviolet-curable resin; and pearl powder dispersed in the ultraviolet-curable resin.

In a preferable embodiment, the pearl powder is at least one selected from the group consisting of powder obtained as a result of pulverizing pearl and powder obtained as a result of pulverizing mother-of-pearl.

In a preferable embodiment, the ultraviolet-curable resin has silver ion incorporated thereinto.

In a preferable embodiment, the silver ion is contained at 10% by mass or less with respect to the entirety of the artificial nail composition.

In a preferable embodiment, the ultraviolet-curable resin has at least one of shellac and hydroxypropylcellulose incorporated thereinto.

In a preferable embodiment, the artificial nail composition contains at least one selected from the group consisting of titanium oxide powder and power stone powder.

In a preferable embodiment, the artificial nail composition is accommodated in a vessel; the vessel includes a vessel main body which is bottle-shaped; the vessel main body is formed of a plastic material; a bottle opening section having an opening through which the artificial nail composition can be introduced is provided in an upper part of the vessel main body; a lid section for closing the opening is attached to the bottle opening section; and an application section for applying the artificial nail composition is attached to the lid section.

In a preferable embodiment, the artificial nail composition is accommodated in a vessel; the vessel is pen-shaped; the vessel includes a cylindrical vessel main body; and the application section for applying the artificial nail composition is attached to a tip part of a conical section extending from the vessel main body.

A method for forming an artificial nail according to the present invention is a method for forming an artificial nail on a nail. The method comprises the steps of applying an artificial nail composition containing an ultraviolet-curable resin and pearl powder dispersed in the ultraviolet-curable resin on a surface of the nail; and curing the artificial nail composition.

In a preferable embodiment, before the step of applying the artificial nail composition on the surface of the nail, a base coat containing at least one of shellac and hydroxypropylcellulose is formed as a base layer for the artificial nail composition.

In a preferable embodiment, the base coat contains silver ion.

In a preferable embodiment, after the step of curing the artificial nail composition, a top coat containing at least one of shellac and hydroxypropylcellulose is formed on a surface of the artificial nail composition.

In a preferable embodiment, the top coat contains silver ion.

A nail covering agent according to the present invention is a nail covering agent for coating a nail. The nail covering agent comprises a coating solution containing at least one of shellac and hydroxypropylcellulose; and silver ion contained in the coating solution.

In a preferable embodiment, the coating solution includes the shellac; and a solvent solution for dissolving the shellac; the solvent solution is ethanol; and the solvent solution does not contain any organic solvent other than ethanol.

In a preferable embodiment, the nail covering agent is accommodated in a vessel; the vessel includes a vessel main body which is bottle-shaped; the vessel main body is formed of a plastic material; a bottle opening section having an opening through which the nail covering agent can be introduced is provided in an upper part of the vessel main body; a lid section for closing the opening is attached to the bottle opening section; and an application section for applying the nail covering agent is attached to the lid section.

In a preferable embodiment, the nail covering agent is accommodated in a vessel; the vessel is pen-shaped; the vessel includes a cylindrical vessel main body; and the application section for applying the artificial nail composition is attached to a tip part of a conical section extending from the vessel main body.

A method for forming a cover film according to the present invention is a method for forming a cover film on a nail. The method comprises the steps of applying a coating solution containing at least one of shellac and hydroxypropylcellulose and also silver ion on a surface of the nail; and drying the coating solution.

In a preferable embodiment, after the step of drying the coating solution, a nail seal is pasted on a surface of the cover film formed of the coating solution.

In a preferable embodiment, after the step of drying the coating solution, a gel nail curable with ultraviolet rays is formed on a surface of the cover film formed of the coating solution.

In a preferable embodiment, in the step of applying the coating solution on the surface of the nail, the coating solution is applied to a surface of an artificial nail.

A nail covering agent according to the present invention is a nail covering agent for forming a cover film for coating a nail, the nail covering agent comprising: a resin material for forming the cover film; and an antibacterial agent contained in the resin material.

In a preferable embodiment, the cover film is one selected from the group consisting of a gel nail, a base coat and a top coat.

### BRIEF DESCRIPTION OF THE DRAWINGS

FIG. 1A and FIG. 1B are each a perspective view showing a structure of a vessel 100 for accommodating an artificial nail composition 10 in an embodiment according to the present invention.

FIG. 2A through FIG. 2C show steps of forming an artificial nail by applying the artificial nail composition 10 in the embodiment according to the present invention.

FIG. 3 shows a structure of a vessel 100 for accommodating the artificial nail composition 10 in an embodiment according to the present invention.

FIG. 4 shows a structure of a vessel 100 for accommodating the artificial nail composition 10 in an embodiment according to the present invention.

FIG. 5A and FIG. 5B are each a perspective view showing a structure of a vessel 200 for accommodating a nail covering agent 110 in an embodiment according to the present invention.

FIG. 6A through FIG. 6C show steps of forming a cover film 115 by applying the nail covering agent 110 in the embodiment according to the present invention.

FIG. 7A through FIG. 7C show steps of pasting a nail seal 60 on a surface of the cover film 115 and applying a second cover film 115b in an embodiment according to the present invention.

### DETAILED DESCRIPTION OF PREFERRED EMBODIMENTS

The present inventor intended to solve problems in the industry of artificial nail (gel nail) formed of a material curable when being irradiated with ultraviolet rays, i.e., propagation of onychomycosis and germs, and growth of molds. One conceivable reason for such problems is that a gap is made between the human nail and the gel nail and germs are propagated in the gap. In the gel nail industry, there are gel nails having a structure in which molds are not likely to grow, but gel nails formed of an antibacterial material are not sold or commonly used. Measures against these problems taken in the gel nail industry are, in actuality, merely cleaning the nail before the gel nail is attached, cleaning the tools, using a gel nail having a structure in which molds are not likely to grow, and using a medicine for onychomycosis. It is conceivable to apply volatile ethanol (or methanol) to a surface of a nail or an artificial nail (gel nail). This method has a sterilization effect at the instance of application, but the components of ethanol or methanol volatilize quickly and disappear. Therefore, the antibacterial effect does not last for more than 24 hours (or 48 hours).

While developing a pearl-colored gel nail of a natural pearl component which contains pearl powder dispersed in an ultraviolet-curable resin, the present inventor found that this gel nail has an antibacterial effect. An artificial nail composition (gel nail composition) containing pearl powder formed of a natural material dispersed in an ultraviolet-curable resin has an antibacterial effect. This means that a gel nail which has a natural pearl color excellent in terms of nail art and an antibacterial characteristic can be produced without using a strong antibacterial agent harmful to the human body. Moreover, the artificial nail composition (gel nail composition) containing pearl powder also has an effect of reducing the amount of chemical substances (volatile organic compounds; VOC). Therefore, the amount of VOC diffused outside from a vessel of the composition according to the present invention can be reduced. This can reduce the possibility that the operator and/or the user is exposed to uncomfortable odor or the risk of allergy. In addition, pearl and mother-of-pearl contain natural calcium. Therefore, as the amount of pearl or mother-of-pearl is increased, the adverse affect on the nail is reduced, and the nail is less damaged than when the conventional gel nail is used.

Japanese Patent Laid-Open Publication No. 2010-195689 proposes an odorless and safe manicure solution which does not contain any organic solvent. This manicure solution contains minute powder of seashell (scallop shell) mixed in an aqueous emulsion with no use of an organic solvent. However, when the present inventor actually used this manicure solution, the manicure solution was transparent rather than pearl-colored, and the durability of the manicure (nail enamel) was as short as less than 2 days. In addition, the above-mentioned publication discloses a manicure solution but does not refer to or mention a gel nail.

The present invention has been made from such a viewpoint. Hereinafter, with reference to the drawings, embodiments of the present invention will be described. In the following drawings, elements having substantially the same function bear the same reference numerals for the sake of simplicity. Elements which are other than those specifically referred to in this specification but are necessary for the present invention may be understood as a design choice by a person of ordinary skill in the art based on the conventional art. The present invention may be carried out based on the disclosure of this specification and the drawings in combination with the technological common knowledge of the art. The present invention is not limited to the following embodiments.

### First Embodiment

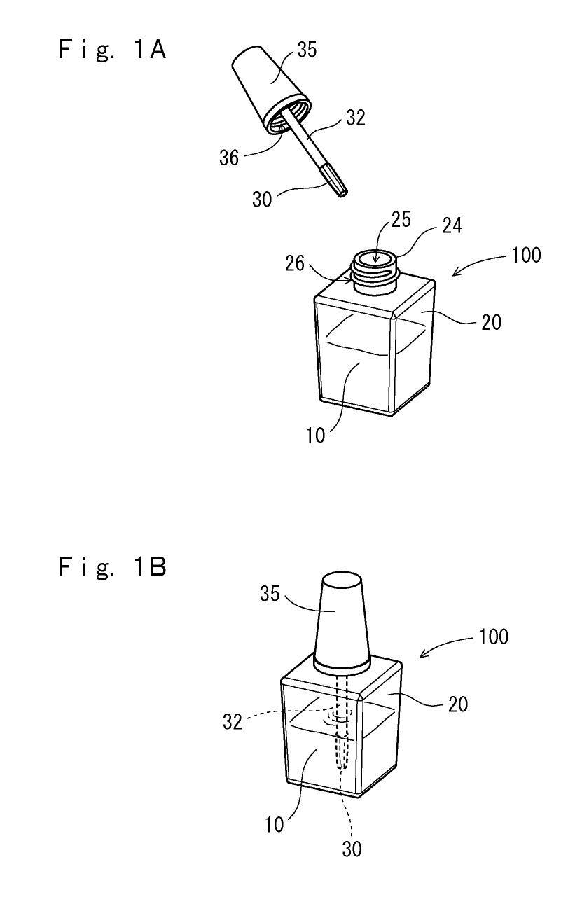

With reference to FIG. 1A and FIG. 1B, a vessel (gel nail vessel) 100 for an artificial nail composition in an embodiment according to the present invention will be described. FIG. 1A and FIG. 1B show a structure of the vessel 100 in this embodiment. FIG. 1A shows a state where a lid section 35 is removed, and FIG. 1B is a state where the lid section 35 is put on.

The vessel (gel nail vessel) 100 in this embodiment accommodates an artificial nail composition 10 for forming an artificial nail. The “artificial nail” in this embodiment is a gel nail formed of a material curable when being irradiated with ultraviolet rays or visible rays. The artificial nail composition 10 in this embodiment is a composition (gel nail composition) which is changed into an artificial nail (gel nail) by being cured when irradiated with ultraviolet rays. A “gel nail” is an artificial nail formed of a gel of a photocurable resin (e.g., acrylic resin) which is curable with ultraviolet rays or visible rays, and is formed by irradiating the gel nail composition applied to a nail with light of an ultraviolet lamp or an LED lamp.

The vessel 100 in this embodiment includes a vessel main body 20 for holding the artificial nail composition (gel nail composition) 10 and an application section 30 for applying the artificial nail composition 10 held in the vessel main body 20 to a nail. The artificial nail composition 10 in this embodiment contains a resin curable with ultraviolet rays or visible rays (ultraviolet-curable resin) and pearl powder dispersed in the resin. In this embodiment, a resin curable with ultraviolet rays and also a resin curable with visible rays are referred to as an “ultraviolet-curable resin”.

The vessel main body 20 shown here is bottle-shaped. A bottle opening section 24 having an opening 25 through which the artificial nail composition 10 can be introduced is formed in an upper part of the vessel main body 20. To the bottle opening section 24, the lid section 35 for closing the opening 25 is attached. The vessel main body 20 in this embodiment is formed of a plastic material (e.g., high density polyethylene, polypropylene, polyethylene terephthalate (PET), etc.) or glass. Preferably, the vessel main body 20 is formed of a plastic material, which is not easily broken.

According to the structure of this embodiment, the application section 30 for applying the artificial nail composition 10 is attached to the lid section 35. The application section 30 is a member like a brush or calligraphy pen. In the example shown here, the application section 30 is attached to a tip part of a support rod 32 extending from an inner top surface of the lid section 35. Namely, the application section 30 is secured to the lid section 35 by the support rod 32 for supporting the application section 30. Therefore, the user can apply a gel nail composition to a nail by holding the lid section 35 with his/her hand and putting the application section 30 located at the tip part of the support rod 32 into contact with the nail.

In the vessel 100 in this embodiment, an outer surface of the bottle opening section 24 and a portion 36 of the lid section 35 corresponding to the outer surface of the bottle opening section 24 have threads 26 and 36, respectively. By inserting the support rod 32 of the lid section 35 into the opening 25 of the bottle opening section 24 and then tightening the lid section 35 to the bottle opening section 24, the vessel 100 (or the vessel main body 20) can be put into a sealed state as shown in FIG. 1B. In this sealed state, the artificial nail composition (gel nail composition) 10 in the vessel main body 20 does not leak outside while the vessel 100 is moved.

In the state shown in FIG. 1B, the application section (or brush section) 30 of the lid section (application tool) 35 is located on an inner bottom surface or the vicinity thereof of the vessel main body 20. The application section 30 is immersed in the artificial nail composition 10. For applying the artificial nail composition 10 to the nail with the lid section (application tool) 35, the lid section 35 is removed from the state shown in FIG. 1B to realize a state shown in FIG. 1A, and the artificial nail composition 10 attached to the application section 30 is applied to the nail.

As described above, the artificial nail composition (gel nail composition) 10 in this embodiment contains an ultraviolet-curable resin curable with ultraviolet rays (or visible rays) and pearl powder dispersed in the ultraviolet-curable resin. In a preferable embodiment, the artificial nail composition 10 does not contain an organic solvent (specifically, any of the specified 54 types of organic solvents generally used in the nail industry) except for a solvent used to dissolve the ultraviolet-curable resin, and the artificial nail composition 10 is formed of only the ultraviolet-curable resin and the pearl powder.

An ultraviolet-curable resin used to form the artificial nail composition (gel nail composition) 10 is, for example, a gel-like resin such as a urethane-acrylic resin or the like. The gel is curable when being irradiated with light of an ultraviolet lamp or an LED lamp. Specific examples of the ultraviolet-curable resin include urethane resins and acrylic resins. A gel nail is formed of a nail material obtained by a photopolymerization reaction, by which a fluid synthetic resin (viscous liquid) applied on a nail is cured when being irradiated with ultraviolet rays (UV-A) or visible rays (Vis). In this embodiment, the term “ultraviolet-curable resin” is used, but precisely, a resin curable with rays in the visible range may also be referred to as an ultraviolet-curable resin, in addition to a resin curable with ultraviolet rays.

The artificial nail composition 10 is cured when being irradiated with ultraviolet rays (or ultraviolet-visible rays) as a result of photopolymerization. A main component of the artificial nail composition 10 is an ultraviolet-curable resin, typically, a urethane resin or an acrylic resin. A specific example thereof is a mixture of urethane acrylate oligomer, epoxy acrylate oligomer, and methacrylic acid ester monomer-acrylic acid oligomer-photoinitiator. The photoinitiator is a photoinitiator for the gel nail, and is a component which starts polymerization by absorbing light (ultraviolet rays, visible rays) and thus generating a radical (unpaired electron).

The pearl powder in this embodiment is formed of pearl particles or mother-of-pearl particles. The pearl powder formed of pearl particles can be produced by use of a machine for pulverizing pearl into minute particles. The particles (minute powder) of mother-of-pearl (e.g., pearl oyster or Pinctada martensii) can be produced by, for example, pulverizing mother-of-pearl. Mother-of-pearl is a shellfish used for generating pearl or cultivated pearl, and exhibits a beautiful shine inside the shell. Examples of the mother-of-pearl include Pinctada martensii, Hyriopsis schlegelii, Pinctada margaritifera, Pinctada maxima, Pteria penguin and the like. The particles of the pearl powder have an average particle diameter of 1 μm to 800 μm (in one example, 300 μm or less, or 3 μm to 300 μm, or about 3 μm), although these are merely examples. The pearl powder may be contained in an ultraviolet-curable resin at a content of about 30% (% by mass) (e.g., 20% to 40%). In the case where the mass of the artificial nail composition 10 may be varied by the content of other components, the pearl powder may be contained at a content of 100 parts by mass or less (e.g., 66 parts by mass or less) or 1 part by mass or greater, where the mass of the ultraviolet-curable resin is 100 parts.

By performing genetic engineering on mother-of-pearl (e.g., pearl oyster), colors such as gold, pink, black, blue, silver and the like can be expressed. Therefore, the artificial nail composition 10 is available in various colors of pearl. The genetic engineering on mother-of-pearl can be conducted using a known technology. By pulverizing the genetic-engineered mother-of-pearl into minute particles, prescribed white pearl powder can be obtained. Mother-of-pearl (e.g., pearl oyster), which would be otherwise disposed of as waste, is effectively utilized. As described above, pearl particles (minute powder) may be incorporated into the ultraviolet-curable resin instead of (or in addition to) the mother-of-pearl powder. In the case where pearls which are of such a shape or quality that cannot be used as jewels and thus are not costly are pulverized and particles of such pearls are used, the cost of the artificial nail composition 10 may not be very high.

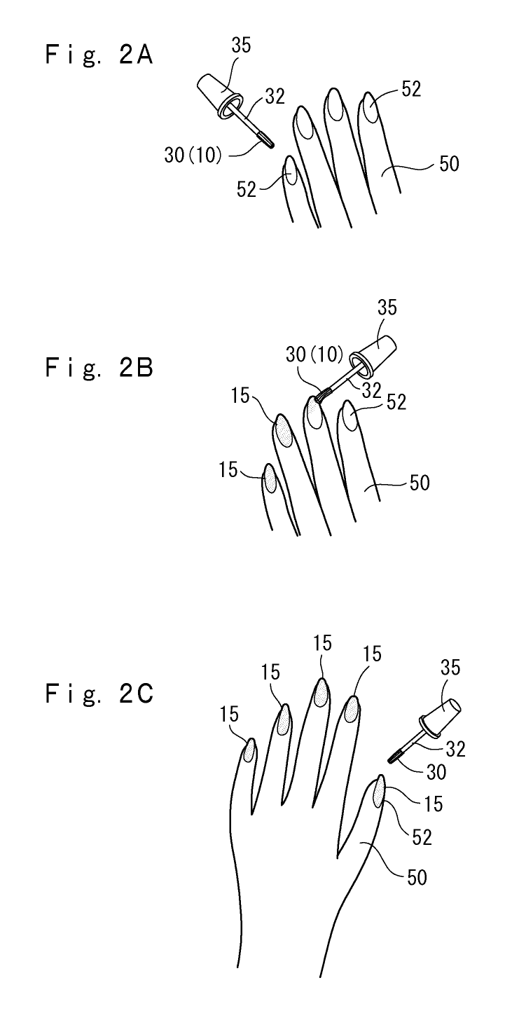

Now, with reference to FIG. 2A through FIG. 2C, steps of applying the artificial nail composition 10 in this embodiment to a nail 52 of a user 50 and thus forming a gel nail (artificial nail) 15 on a surface of the nail 52 will be described.

First, as shown in FIG. 2A, the artificial nail composition 10 in this embodiment is attached to the tip part (application part) 30 of the application tool (lid section) 35 like a brush or calligraphy pen, and the artificial nail composition 10 attached to the tip part 30 of the application tool 35 is brought close to the nail 52 of the user 50. The artificial nail composition 10 in this example is accommodated in the vessel 100 shown in FIG. 1A.

Next, as shown in FIG. 2B, the artificial nail composition 10 attached to the tip part 30 of the application tool 35 is applied to the nail 52. A gel-like ultraviolet-curable resin for forming the artificial nail composition 10 is located on the nail 52, and thus cover film 15 is formed. As shown in FIG. 2C, after the artificial nail composition 10 is applied to a fingernail to which the artificial nail composition 10 is to be applied (herein, the nails 52 of all the fingers), the cover film 15 is irradiated with light of an ultraviolet lamp. Thus, the cover film 15 (i.e., the artificial nail composition 10) can be cured. Namely, the gel nail (artificial nail) is formed. The thickness of the cover film 15 depends on, for example, the concentration of the solution and the amount of the artificial nail composition 10 used, and is, for example, 5 mm or less (in one example, about 5 mm to 0.01 mm).

The artificial nail composition 10 in this embodiment contains pearl powder dispersed in the ultraviolet-curable resin. Therefore, the cover film 15 and the gel nail obtained as a result of the cover film 15 being cured express a pearl color. This pearl color is the color of naturally occurring pearl, and is different from the color of fake pearl.

The concentration of the pearl powder in the artificial nail composition 10 in this embodiment is, for example, 0.1 to 50% (% by mass), and the remaining part is the ultraviolet-curable resin. In other embodiments, the artificial nail composition 10 may contain at least one of a scent agent and an antibacterial agent in addition to the ultraviolet-curable resin. The artificial nail composition 10 may contain a pigment for coloring the gel nail. For example, a pigment (including lame for providing a shine or gloss, such as isinglass or mica, etc.) may be incorporated, so that the cover film 15 formed of the artificial nail composition 10 is colored and transparent, colored and opaque or the like, instead of being pearl-colored. The artificial nail composition 10 contains a component which vaporizes after being applied. Therefore, the concentration of the pearl powder in the state of the artificial nail composition 10 is merely exemplary. There is no specific limitation on the concentration as long as the cover film 15 is formed. The artificial nail composition 10 may also contain another functional substance (e.g., substance such as a scent agent which releases scent, etc.).

The artificial nail composition 10 may be applied to all the nails 52 or only a part of the nails 52. The artificial nail composition 10 may be applied to the entire area of one nail 52 or a part of the area of one nail 52 (e.g., only a tip part of the nail 52). The artificial nail composition 10 may be applied to the nail 52 in the state where a base coat has already been applied to the nail 52. On a surface of the gel nail obtained as a result of the artificial nail composition 10 being cured, a top coat may be applied.

In addition to the gel nail (or the cover film 15) formed of the artificial nail composition 10, a nail stone (not shown) may be attached. A nail stone can be attached to a surface of the cover film 15. According to an easy method of attaching a nail stone, the nail stone is put on the pre-curing cover film 15 in a gel state and then the cover film 15 is cured. Nail stones are available in various materials and types and also in various shapes and colors. There is no specific limitation on the type or shape of the nail stones. Examples of the nail stones include Swarovski stones (stones produced by Swarovski AG), acrylic or glass stones (stones formed of acrylic resins or glass), pearl stones (stones processed to be like pearls; semispherical acrylic stones processed to have a pearl color), Bullion (small spherical particle-type stones) and the like.

The present inventor performed an antibacterial test on the artificial nail composition 10 in this embodiment. The artificial nail composition 10 was recognized to have an antibacterial characteristic. The content (% by mass) of the pearl powder in the artificial nail composition 10 in this test example was 50% or less, and specifically, 20%. The test was performed by a method conformed to JIS Z 2801²⁰¹⁰, 5. As the bacteria, Staphylococcus aureus NBRC 12732 and Escherichia coli NBRC 3972 were supplied. The artificial nail composition 10 in this embodiment was recognized to be antibacterial against both of Staphylococcus aureus and Escherichia coli. As a non-processed test piece, a polyethylene film was used.

Regarding Staphylococcus aureus, in the case of the artificial nail composition 10 in this embodiment, the common logarithm value of live bacteria was <−0.20, and the antibacterial activity value was >4.8. Thus, the artificial nail composition 10 was recognized to be antibacterial against Staphylococcus aureus. In the case of the non-processed test piece provided as a reference example, the common logarithm value of live bacteria was 4.1 immediately after inoculation of a bacterium solution and 4.7 after the bacteria were cultivated for 24 hours. The non-processed test piece was not recognized to be antibacterial against Staphylococcus aureus. Regarding Escherichia coli, in the case of the artificial nail composition 10 in this embodiment, the common logarithm value of live bacteria was 3.6, and the antibacterial activity value was >2.1. Thus, the artificial nail composition 10 was recognized to be antibacterial against Escherichia coli. In the case of the non-processed test piece provided as a reference example, the common logarithm value of live bacteria was 4.1 immediately after inoculation of a bacterium solution and 5.8 after the bacteria were cultivated for 24 hours. The non-processed test piece was not recognized to be antibacterial against Escherichia coli. As the reference for approving that a test sample is antibacterial, an antibacterial activity value (bacteriostatic activity value) of 2.1 or greater can be used.

Since the artificial nail composition 10 in this embodiment has an antibacterial characteristic, the cover film 15 (and the cured gel nail) formed of the artificial nail composition 10 in this embodiment can be provided with an antibacterial characteristic. Therefore, an artificial nail (gel nail) formed of the artificial nail composition 10 in this embodiment can suppress propagation of bacteria (germs) owing to the antibacterial effect. Namely, when the artificial nail composition 10 in this embodiment is used, propagation of germs on the surface of the nail 52 (surface of the gel nail) can be suppressed or prevented. Since the nail 52 (surface of the gel nail) may touch the mouth or an area around the eyeballs, it is not hygienically preferable that germs are propagated on the surface of the nail 52. Especially, scratching an eye with an unhygienic nail may cause an eye disease. Therefore, providing the gel nail in this embodiment with an antibacterial characteristic to prevent propagation of germs on the nail 52 is highly advantageous hygienically.

The pearl powder dispersed in the ultraviolet-curable resin of the artificial nail composition 10 is a naturally derived component which is not harmful to the human body and thus is highly safe. Therefore, the artificial nail composition 10 containing the pearl powder does not have an adverse affect on the human body beyond the influence (adverse affect) exerted on the human body by the ultraviolet-curable resin for forming the artificial nail composition 10. It is not guaranteed that germs other than Escherichia coli and Staphylococcus aureus are never attached or propagated. In order to suppress unexpected attachment or propagation of germs, it is preferable that the powder is incorporated after being sterilized by gas sterilization or the like.

The artificial nail composition 10 in this embodiment is used as a main part of the gel nail. Alternatively, the artificial nail composition 10 may be used as a base coat and/or a top coat, utilizing the antibacterial characteristic of the artificial nail composition 10. In the above, the artificial nail composition 10 is mainly applied to the nail 52 of a hand. The present invention is not limited to this. The artificial nail composition 10 may be applied to a nail of a foot. Alternatively, the artificial nail composition 10 may be applied to an artificial nail (fake nail, etc.) as well as the human nail. The post-curing cover film 15 formed of the artificial nail composition 10 may be attached to the human nail as a unique decorated fake nail.

In addition, in order to improve the antibacterial characteristic of the artificial nail composition 10 in this embodiment, another antibacterial material may be incorporated into the artificial nail composition 10. Examples of such an antibacterial material include silver ion (Ag⁺), titanium oxide and the like. In a preferable embodiment, the artificial nail composition 10 contains an ultraviolet-curable resin, pearl powder, and an antibacterial material (silver ion) (a pigment may be optionally incorporated). By incorporating another antibacterial material into the artificial nail composition 10, the cover film 15 (gel nail) formed of the artificial nail composition 10 can be stably provided with an antibacterial characteristic even when the type of the ultraviolet-curable resin is changed (especially when a novel ultraviolet-curable resin different from the conventional ultraviolet-curable resin is used) or even when the amount of the pearl powder is changed.

An artificial nail composition 10 containing silver ion can suppress propagation of bacteria (germs) owing to the antibacterial effect of the silver ion. The silver ion (Ag⁺) having an antibacterial characteristic may be used in the form of, for example, silver ion water, silver nanoparticles or the like. A silver ion (Ag⁺) refers to a silver ion existing as a cation as a result of electrons being removed from silver (Ag) as an atom. The size of a silver ion is substantially the same as that of a silver atom and is about 200 picometers, which is the so-called atom size. Silver ion can be eluted, namely, generated by, for example, performing electrolysis of silver in water. In the case of silver ion water, silver ion is independently free in molecules of water and is basically present in an ion state in water. There are two main methods for producing silver ion; namely, an electrolysis method of flowing an electric current through a pure silver electrode in water to generate silver ion, and a chemical method of adding a chemical containing a silver ion component to water. Silver nanoparticles are obtained by putting silver into particles in the order of nanometers. Silver nanoparticles retaining silver ion obtained by putting alumina silica, which is a mineral, into minute particles can be used.

Silver ion is strongly adsorbed to cell of various types of bacteria and can block cell enzyme of the bacteria to extinguish the bacteria. Therefore, propagation of bacteria can be controlled by silver ion. Silver ion has a characteristic of providing a relatively high antibacterial effect while being highly safe to the human body. Accordingly, even if the artificial nail composition 10 or the gel nail inadvertently enters the mouth of a human, silver ion causes substantially no problem. Both of silver ion and pearl powder are highly safe to the human body, which is preferable.

According to the structure of this embodiment, the content of silver ion (antibacterial material) may be 10% (% by mass) or less, typically, 5% (% by mass) or less (e.g., 2 to 3% by mass, or about 5% by mass) in the artificial nail composition 10. In order to increase the antibacterial effect provided by the silver ion, it is conceivable to incorporate silver ions in a large amount. However, when silver ion is incorporated at a content of 20% by mass or greater (occasionally, greater than 10% by mass, or greater than 5% by mass), the color of the incorporated silver ion may influence the color of the artificial nail (gel nail). Therefore, the amount of silver ion may be less than 20% by mass, for example, 10% by mass or less, or 5% by mass or less. In the case where there is no problem of color, an appropriate amount of silver ion (occasionally, greater than 10% by mass) may be incorporated.

In the above silver ion (Ag⁺) is used as an antibacterial material. Alternatively, another antibacterial material may be incorporated into the artificial nail composition 10 under the condition of providing an antibacterial effect. For example, titanium oxide may be incorporated instead of (or in addition to) silver ion. Still alternatively, silver ion, titanium oxide, and another antibacterial material (e.g., catechin, chitosan, hinokitiol) may be used. Titanium oxide may be incorporated into the artificial nail composition 10 for the purpose of exhibiting a characteristic of blocking UV (ultraviolet rays). Examples of the another antibacterial material include inorganic antibacterial agents (using the bacteriostatic function of metal ion), and organic antibacterial agents (antibacterial agents using organic substances; synthetic or natural antibacterial agents).

The vessel 100 in this embodiment includes the vessel main body 20 for holding the artificial nail composition 10 and the application section 30 for applying the artificial nail composition 10. The artificial nail composition 10 contains pearl powder dispersed in an ultraviolet-curable resin. Therefore, a pearl-colored artificial nail (gel nail) formed of pearl powder can be formed by the application section 30 in the vessel 100 in this embodiment. The artificial nail (gel nail) formed of an ultraviolet-curable resin having pearl powder dispersed therein exhibits an antibacterial characteristic, and therefore can suppress propagation of germs or growth of molds which occurs in the state where the artificial nail (gel nail) is formed. Pearl and mother-of-pearl contain natural calcium. Therefore, as the amount of pearl or mother-of-pearl is increased, the adverse affect on the nail is decreased, and thus the artificial nail composition 10 makes the nail less likely to be damaged than the conventional gel nail.

In the field of artificial nail (gel nail) formed of a material curable when being irradiated with ultraviolet rays or visible rays, there may be problems of propagation of onychomycosis and germs, and growth of molds. Currently, gel nails formed of an antibacterial material are not sold or commonly used. According to the structure of this embodiment, the artificial nail composition 10 containing pearl powder is used to form a gel nail having a color of natural pearl and also having an antibacterial characteristic. The problems of propagation of onychomycosis and germs, and growth of molds, are considered to occur because germs are propagated in a gap made between the human nail and the gel nail. According to the structure of this embodiment, the artificial nail composition (gel nail composition) 10 itself has an antibacterial characteristic, and therefore can suppress propagation of germs (or molds) in such a gap. Accordingly, the present invention can provide a more direct and simpler measure against the problems of growth of molds and the like, unlike the measures taken conventionally in the gel nail industry, for example, cleaning the nail before the gel nails is attached, cleaning the tools, using a gel nail having a structure in which molds are less likely to grow, using a medicine for onychomycosis, or the like.

The present inventor also confirmed by experiments that the artificial nail composition 10 containing pearl powder (e.g., powder derived from Pinctada maxima) also has an effect of decreasing a chemical substance (volatile organic substance). The effect of decreasing the amount of a chemical substance can be confirmed by performing a formaldehyde test on the artificial nail composition 10 in this embodiment, specifically by the following method. The artificial nail composition 10 is extracted for 1 hour with 100 ml of purified water of 40° C. and treated by a method conformed to JIS L-1041:2000 (resin-treated textile and test method for the textile, 6.3 Free formaldehyde test, 6.3.1 Liquid phase extraction method, b) Acetylacetone method, Method B. The absorbance of the resultant artificial nail composition 10 is measured.

Since the artificial nail composition 10 in this embodiment can suppress generation of a chemical substance (volatile organic compound), the amount of volatile organic compound going outside the vessel 100 in this embodiment can be decreased. As a result, the degree of discomfort felt by the operator applying the artificial nail composition 10 to the nail 52 and/or the user having the nail 52 to which the artificial nail composition 10 is applied can be reduced.

In addition, the artificial nail composition 10 may contain a far-infrared radiation material for emitting far-infrared rays. Examples of the far-infrared radiation material include ceramics and charcoal (bincho charcoal, etc.). By incorporating the far-infrared radiation material into the artificial nail composition 10, the gel nail formed of the artificial nail composition 10 is caused to emit far-infrared rays. Thus, an effect of promoting blood circulation by the far-infrared rays can be provided. According to the structure of this embodiment, the far-infrared radiation material may be incorporated at a content of about 10% (% by mass) (e.g., 5% to 20%) in the artificial nail composition 10 (or the cover film 15).

The artificial nail composition 10 may contain a power stone powder obtained by pulverizing power stone such as natural rock or the like can be incorporated into. A power stone is a type of jewel (precious stone, semiprecious stone) which is considered to have a certain type of special power. The concept of “power stone” basically encompasses jewels, but in addition, biological materials such as tortoiseshell, ivory and the like, noble metals such as gold, silver and the like, base metal such as copper and the like, fossils, food materials such as rock salt and the like may be used as a power stone. Examples of the power stone powder which may be incorporated into the artificial nail composition 10 in this embodiment include jade powder, crystal powder and the like. When a gel nail formed of the artificial nail composition 10 containing such power stone powder is used, the nail 52 can be coated with power stone. The power stone powder can also be used as a pigment owing to the color thereof. For example, by incorporating gold powder into the artificial nail composition 10, a pearl-colored gel nail containing gold powder can be formed.

The artificial nail composition 10 may also contain a scent agent. In the case where an ultraviolet-curable resin, for forming the artificial nail composition 10, containing a scent agent does not emit any scent, a technique of dispersing nanocapsules (containing a scent agent in capsules) which are not dissolved in an organic solvent and emit a scent when receiving a physical pressure may be used. Even when a liquid scent agent is dispersed in an ultraviolet-curable resin, the artificial nail composition 10 may not emit any scent. However, when such nanocapsules are used, the capsules are broken on the surface of the nail 52 and a scent can be emitted.

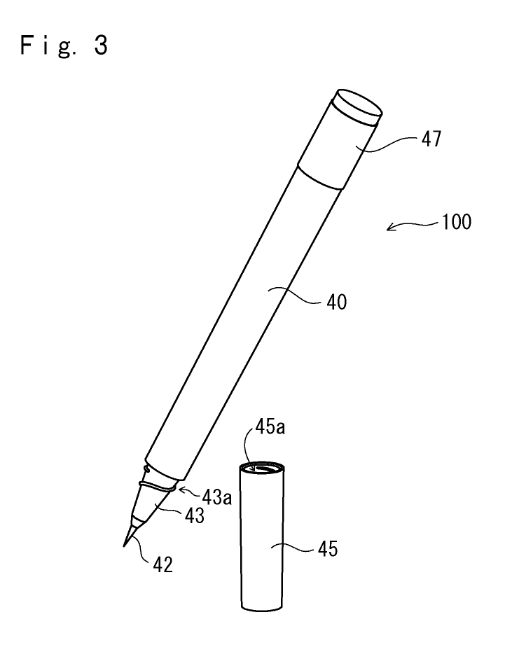

In the above embodiment, the vessel 100 has the structure as shown in FIG. 1A and FIG. 1B. The vessel according to the present invention is not limited to this. For example, the application section 30 is not necessary attached to a part of the lid section 35 of the vessel 100. The application section 30 may be provided separately from the lid section 35. The vessel 100 according to the present invention may be pen-shaped as shown in FIG. 3.

The vessel 100 shown in FIG. 3 includes a cylindrical vessel main body 40 for accommodating the artificial nail composition 10. An application section 42 is located at a tip part of a conical section 43 extending from the vessel main body 40. In this example, the application section 42 is like a brush at the tip of a calligraphy pen. The inside of the vessel main body 40 and the application section 42 are continuous to each other, and the artificial nail composition 10 accommodated in the vessel main body 40 is supplied to the application section 42 sequentially. The artificial nail composition 10 can be applied to the nail 52 with the application section 42 to which the artificial nail composition 10 is sequentially supplied.

At a rear end of the vessel main body 40, a sealing section (closed-bottom cylindrical member) 47 for closing a rear end opening of the vessel main body 40 is provided. When the sealing section 47 is removed, the rear end opening of the vessel main body 40 is exposed, and the artificial nail composition 10 can be supplied from the rear end opening. In the example shown here, a cap section 45 for accommodating the conical section 43 and the application section (brush section) 42 is attachable. In this example, an outer surface of the conical section 43 and an inner surface of the cap section 45 respectively have threads 43a and 45a. By rotating the cap section 45 with respect to the conical section 43, the cap section 45 and the conical section 43 are put into engagement with each other.

For securing the cap section 45, the cap section 45 may be inserted into the vessel main body 40 along a longitudinal axial direction thereof, instead of being rotated and thus engaged with the conical section 43. For securing the sealing section 47 to the vessel main body 40, the sealing section 47 may be rotated along threads or another technique (e.g., technique of inserting the sealing section 47 into the vessel main body 40 along the longitudinal axial direction thereof) may be used. The vessel 100 shown in FIG. 3 has the cylindrical vessel main body 40. Alternatively, the vessel main body 40 may have another shape (e.g., hexagonal prism).

For the pen-type vessel 100 shown in FIG. 3, the member of the supply path from the vessel main body 40 to the application section 42 (e.g., porous member) needs to be designed such that the pearl powder contained in the artificial nail composition 10 is continuously supplied to the application section 42. Pearl powder having a particle diameter of 300 μm or less is easily supplied to the application section 42 because such a particle diameter is of the same level as that of a pigment. In an example of this embodiment, the particle diameter of the pearl powder may be about 3 μm. In this case, the member of the supply path for supplying the pearl powder from the vessel main body 40 to the application section 42 can be easily produced. The vessel main body 40 in this embodiment is formed of a resin. The vessel main body 40 may be transparent so that the artificial nail composition 10 accommodated therein can be seen. Alternatively, the vessel main body 40 may be formed of an opaque material except for a partial transparent section, so that a remaining amount of the artificial nail composition 10 is found.

This pen-type vessel 100 can apply the artificial nail composition 10 to the nail 52 continuously, which is convenient. The vessel 100 is thin and lengthy and thus does not require a large accommodation space, which is convenient for storage and carrying.

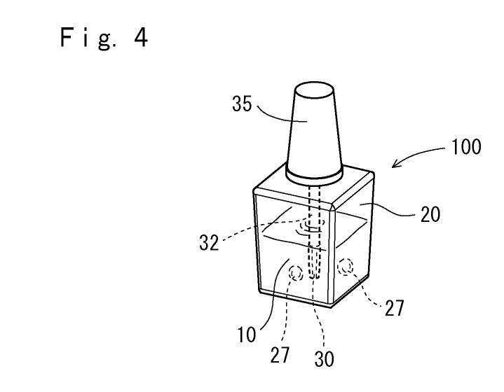

As shown in FIG. 4, pearls 27 may be put inside the vessel main body 20 in the vessel 100 shown in FIG. 1A and FIG. 1B. By putting the pearls 27 in this manner, the pearl powder is dispersed well in the artificial nail composition 10, and also the external appearance of the vessel 100 is improved. Instead of the pearls 27, power stones or the like may be put in the vessel main body 20.

### Second Embodiment

Recently, nail art of decorating nails of hands or feet is a target of attention as a type of fashion especially among women. Methods of decorating nails of hands or feet include, for example, a method of directly putting a color or pattern on the nail with manicure or the like, a method of directly pasting a decorated artificial nail to the nail, and the like. Regarding the method of directly putting a color or pattern on the nail with manicure or the like, there is a limit of technique when a layperson decorates his/her own nail. When such a layperson has a specialist decorate the nail, it is costly and time-consuming. The method of directly pasting a decorated artificial nail to the nail is less costly than having a specialist decorate the nail, and can be done by a layperson easily. However, since the artificial nail easily comes off, there are problems that the user has trouble in everyday life and also the user feels uncomfortable because the artificial nail does not fit well to his/her own nail.

In such a situation, a method of pasting a decorated nail seal to the nail is known (see, for example, Japanese Patent Laid-Open Publication No. 2010-250138). This method is not costly and is easy, and the user can feel that the seal is fit to his/her own nail. Since the pattern of a nail seal is printed with ink or the like, there is a problem that the pattern is poor in water resistance. A nail seal, which is easy to paste even for a layperson and low-cost and makes the user feel that the nail seat fits well to his/her own nail, is now becoming increasingly popular. However, as a result of studying the nail seal, the present inventor found the following problems in addition to the poor water resistance.

When the nail seal is directly pasted to the nail and left on the nail for about 2 weeks, the nail seal sticks to the nail and cannot be removed easily. In addition, since the adhesive on the nail seal is pasted on the human nail for a long time, the color of the human nail may become cloudy. However, if the viscosity of the nail seal is decreased, the possibility that the nail seal is not pasted well to the nail is high. Therefore, when the nail seal is directly pasted on the nail, the user needs to accept the inconveniences that the nail seal is difficult to be removed and the human nail becomes cloudy.

In order to suppress the human nail from becoming cloudy due to the adhesive of the nail seal, the present inventor examined use of shellac. Shellac is a substance used for food, which is tasteless, odorless and harmless to the human body, and thus is highly safe. The present inventor considered that coating a surface of the human nail with a nail covering agent (coating agent) containing shellac as a main component could protect the nail, unlike the case where the adhesive of the nail seal is directly pasted on the human nail; and carried it out. The result was successful.

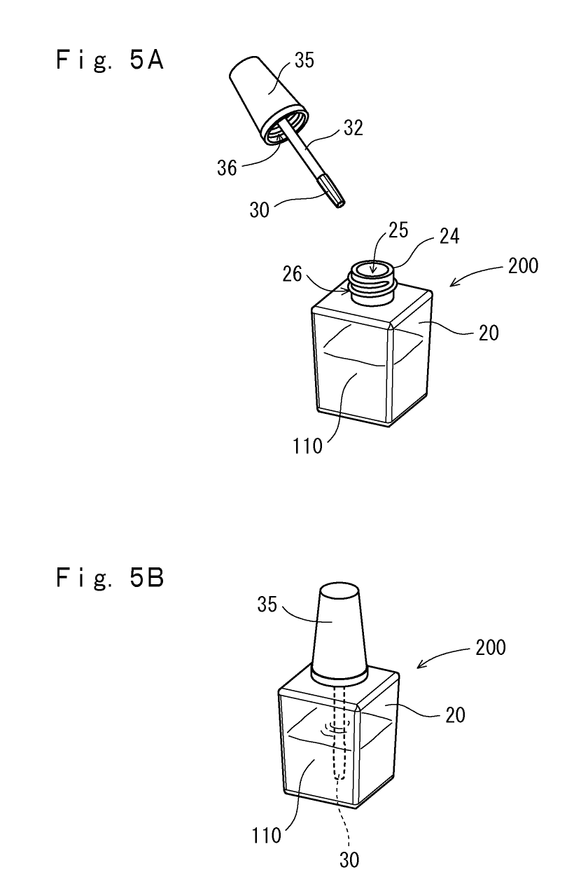

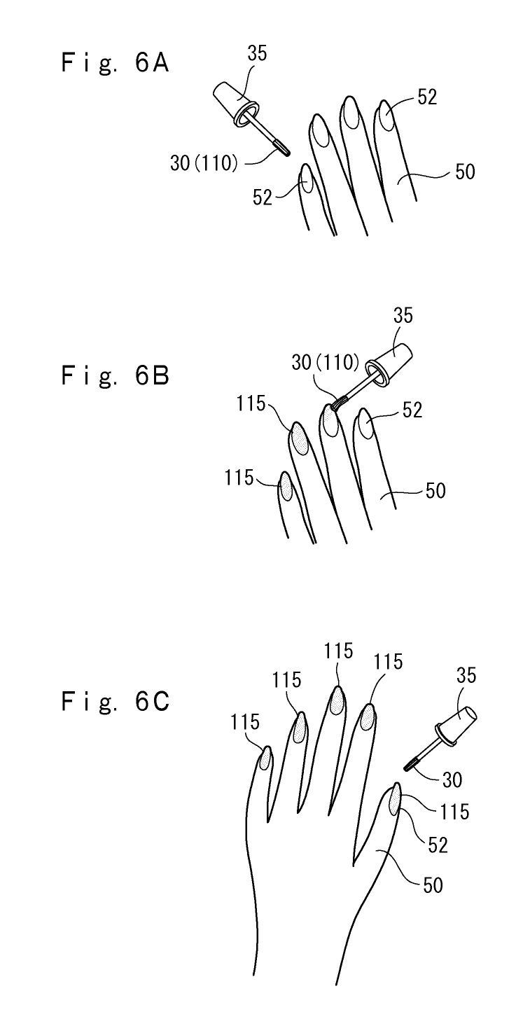

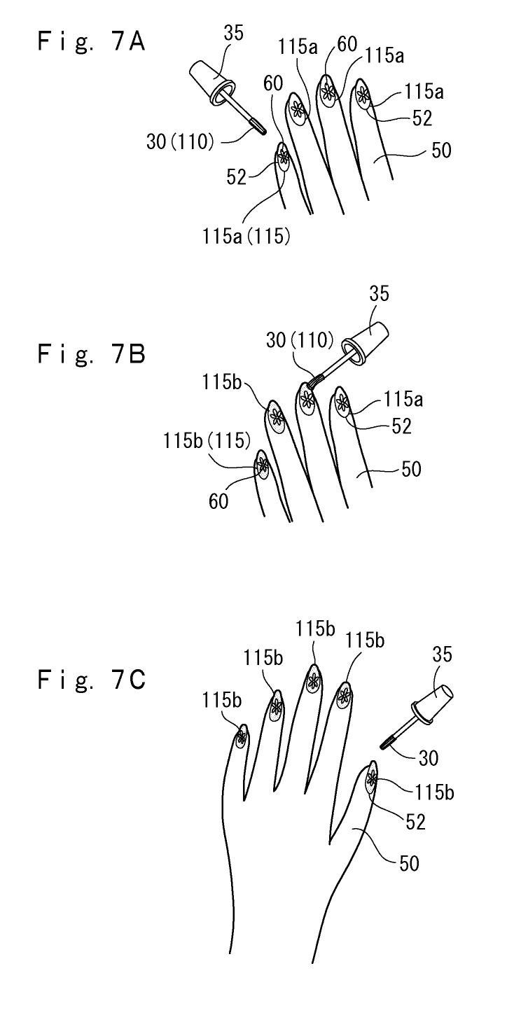

With reference to FIG. 5A, FIG. 5B, FIG. 6A through FIG. 6C and FIG. 7A through FIG. 7C, a nail covering agent 110 in an embodiment according to the present invention will be described. FIG. 5A and FIG. 5B show a vessel 200 accommodating the nail covering agent 110 in this embodiment. FIG. 5A and FIG. 5B correspond to FIG. 1A and FIG. 1B and will not be described here. The nail covering agent 110 in this embodiment may be accommodated in the vessel 100 shown in FIG. 3 or FIG. 4.

FIG. 6A through FIG. 6C show steps of applying the nail covering agent 110 in this embodiment to form a cover film 115. The nail covering agent 110 in this embodiment is a nail covering agent for coating a nail, and contains shellac and a solvent solution for dissolving shellac (e.g., ethanol). The nail covering agent 110 in this embodiment contains shellac as a main component.

Shellac is a resin-like substance obtained by refining an insect covering substance secreted by Laccifer lacca KERR and several types of Laccifer lacca close thereto. Shellac is a transparent solid which has a color in the range from yellow to brown at room temperature and becomes white and transparent when being refined. Shellac is tasteless, odorless and harmless to the human body. Shellac is soluble in an alcoholic solvent and is resistant against other organic solvents. When an alcoholic solution or an aqueous solution of shellac is vaporized, a transparent cover film formed of shellac can be formed.

The nail covering agent 110 in this embodiment is formed into the cover film 115 as follows. An alcoholic solution containing shellac dissolved therein is applied to a surface of a nail 52, and the alcohol in the solution is vaporized. As a result, the cover film 115 formed of shellac (cover film formed of the nail covering agent) is formed. Specifically, the application is performed in the steps shown in FIG. 6A through FIG. 6C.

First, as shown in FIG. 6A, the nail covering agent 110 in this embodiment is attached to the tip part 30 of the application tool 35 like a brush or calligraphy pen, and the nail covering agent 110 attached to the tip part 30 of the application tool 35 is brought close to the nail 52 of the user 50. The nail covering agent 110 in this example is accommodated in the bottle 20 (200) corresponding to the application tool 35. The concentration of shellac in the nail covering agent 110 in this embodiment is, for example, 1 to 60% (% by mass). When the cover film 115 is to be formed thick, the concentration of shellac in the nail covering agent 110 is preferably 20 to 60% (% by mass). When the cover film 115 is to be formed thin, the concentration of shellac in the nail covering agent 110 is preferably 1 to 20% (% by mass). The remaining part is the solvent solution (alcohol). Since the solvent solution is vaporized after being applied, the concentration of shellac in the nail covering agent 110 is merely exemplary. There is no specific limitation on the concentration as long as the cover film 115 is formed.

The solvent solution of the nail covering agent 110 in this embodiment is ethanol, and the nail covering agent 110 in this example does not contain any organic solvent other than ethanol. Namely, any of the specified 54 types of organic solvents generally used in the nail industry is not used. Therefore, neither the operator nor the consumer absorbs the volatile organic compound (VOC) having a specific odor which is generated when any of the specified 54 types of organic solvents is used. No harm is done to the human body, or there is no risk of allergy, due to any of about 54 types of the organic solvents. In addition, the ethanol solution containing shellac (or occasionally, an aqueous solution containing shellac) is not harmful, and thus can guarantee safety to the operator and the consumer as opposed to any organic solvent usually used.

Next, as shown in FIG. 6B, the nail covering agent 110 attached to the tip part 30 of the brush 35 is applied to the nail 52. When the solvent solution (alcohol) of the nail covering agent 110 is vaporized and the nail covering agent 110 is dried, the cover film 115 in this embodiment is formed. Herein, the term “dried” means that the nail covering agent 110 is completely dried and also that a part of the solvent solution remains. It is sufficient that the solvent solution of the nail covering agent 110 is vaporized to such a degree that the cover film 115 is formed. The thickness of the cover film 115 depends on, for example, the concentration of the solution and the amount of the nail covering agent 110 used, and is, for example, 0.5 mm or less (in one example, about 0.3 mm to 0.01 mm).

As described above, as shown in FIG. 6C, the cover film 115 in this embodiment is formed on the nail 52 to which the nail covering agent 110 is to be applied (herein, the nails of all the fingers). After this, a nail seal (not shown) may be pasted to a surface of the cover film 115.

The nail seal includes a main layer having a printed pattern and a viscous layer formed of a viscous material provided on a rear surface of the main layer. The cover film 115 in this embodiment is present between the viscous layer of the nail seal and the human nail, and thus can prevent the human nail from becoming cloudy due to the viscous layer of the nail seal. The cover film 115 allows the nail seal to be removed easily and neatly. In addition, the nail covering agent 110 and the cover film 115 in this embodiment do not contain any organic solvent (i.e., organic solvent other than alcohol). Therefore, the influence that the human nail is damaged (e.g., becomes cloudy, etc.) due to the organic solvent can be prevented.

Next, with reference to FIG. 7A through FIG. 7C, steps of pasting a nail seal 60 on the cover film 115 (first cover film 115a) and then applying the cover film 115 (second cover film 115b) will be described.

First, after the state shown in FIG. 6C, the nail seal 60 is pasted on the cover film (first cover film) 115a. The nail seal 60 in this example has a pattern of a flower, but the nail seal 60 may have any pattern. The nail seal 60 may be pasted so as to cover the entire nail 52 or a part of the nail 52.

Together with the nail seal 60, a nail stone (not shown) may be attached. In the case where the nail seal 60 is pasted so as to cover a part of the nail 52, the nail stone may be attached to a surface of the nail seal 60 or to a surface of the cover film 115a. Nail stones are available in various materials and types and also in various shapes and colors. There is no specific limitation on the type or shape of the nail stones. Examples of the nail stones include Swarovski stones (stones produced by Swarovski AG), acrylic or glass stones (stones formed of acrylic resins or glass), pearl stones (stones processed to be like pearls; semispherical acrylic stones processed to have the pearl color), Bullion (small spherical particle-type stones) and the like.

Next, as shown in FIG. 7A, like in FIG. 6A, the nail covering agent 110 is attached to the tip part 30 of the application tool (brush) 35, and the nail covering agent 110 attached to the tip part 30 of the application tool 35 is brought close to the nail 52. Below the nail seal 60, the cover film 115a is formed as a base layer.

Next, as shown in FIG. 7B, the nail covering agent 110 attached to the tip part 30 of the brush 35 is applied to the surface of the nail seal 60 (or a surface of the nail 52 having the nail seal 60 pasted thereon). Like in FIG. 6B, when the solvent solution (alcohol) of the nail covering agent 110 is vaporized and the nail covering agent 110 is dried, the cover film 115b (second cover film) in this embodiment is formed.

In this manner, as shown in FIG. 7C, the cover film (second cover film) 115b in this embodiment is formed on the nail 52 to which the nail covering agent 110 is to be applied (herein, the nails of all the fingers). The first cover film 115a may be referred to a “base coat”, and the second cover film 115b may be referred to a “top coat”. The printed pattern on the surface of the nail seal 60 may be poor in durability occasionally. The second cover film 115b (top coat) in this embodiment has an effect of protecting the pattern of the nail seal 60.

The nail covering agent 110 for forming the first cover film 115a and the nail covering agent 110 for forming the second cover film 115b may be the same or different. In consideration of the work efficiency of nailing, it is more convenient that the same nail covering agent 110 is used. From the state shown in FIG. 7C, a nail stone may be attached and the nail covering agent 110 may be applied to cover the nail stone to form a cover film 115. Alternatively, the nail covering agent 110 may be applied so as to avoid the nail stone.

The above-described nail covering agent 110 contains only shellac and a solvent solution for dissolving shellac (e.g., ethanol). The expression that “contains only shellac and a solvent solution (ethanol)” does not exclude the case where another component (e.g., water) is contained at a content of about 5% (% by mass). According to the structure of this embodiment, any other additive may be added. For example, a pigment (including a pigment of lame) may be incorporated, so that the cover film 115 formed of the nail covering agent 110 is colored and transparent, colored and opaque or the like, instead of colorless and transparent. In the case where the cover film 115 contains shellac as a main component, the content of shellac in the cover film 115 is, for example, 50% or greater (% by mass). Especially when the nail covering agent 110 is formed of substantially only shellac, the content of shellac is 98% or greater (% by mass).

In addition, the nail covering agent 110 may contain an antibacterial material. Examples of the antibacterial material include silver ion (Ag⁺), titanium oxide and the like. In a preferable embodiment, the nail covering agent 110 contains shellac and an antibacterial material (silver ion) (a pigment may be optionally incorporated). By incorporating such an antibacterial material into the nail covering agent 110, the cover film 115 formed of the nail covering agent 110 can be provided with an antibacterial characteristic. The cover film 115 containing silver ion can suppress propagation of bacteria (germs) owing to the antibacterial effect of the silver ion in the cover film 115.

In the case where the first cover film 115a as the base coat has an antibacterial characteristic, propagation of germs on the human nail 52 can be suppressed or prevented. In the case where the nail seal 60 is pasted for a long time (e.g., for 2 weeks), providing the first cover film 115a with an antibacterial characteristic to prevent propagation of germs on the human nail 52 is highly advantageous hygienically.

In the case where the second cover film 115b as the top coat has an antibacterial characteristic, propagation of germs on the surface of the nail 52 (i.e., the surface of the nail seal 60) can be suppressed or prevented. Since the nail 52 (surface of the nail seal 60) may touch the mouth or an area around the eyeballs, it is not hygienically preferable that germs are propagated on the surface of the nail 52. Especially, scratching an eye with an unhygienic nail may cause an eye disease. Therefore, providing the second cover film 115b with an antibacterial characteristic to prevent propagation of germs on the nail 52 (surface of the nail seal 60) is highly advantageous hygienically.

Silver ion (Ag⁺) having an antibacterial characteristic may be used in the form of, for example, silver ion water, silver nanoparticles or the like, as described above. Silver ion has a characteristic of providing a relatively high antibacterial effect while being highly safe to the human body. Accordingly, even if the cover film 115 or the nail covering agent 110 inadvertently enters the mouth of a human, silver ion causes substantially no problem. Since both of silver ion and shellac are highly safe to the human body, the cover film 115 containing silver ion and shellac is preferable. According to the structure of this embodiment, the content of silver ion (antibacterial material) may be 10% (% by mass) or less, typically, 5% (% by mass) or less or about 5% in the artificial nail composition 10. The amount of silver ion may be determined referring to the above description regarding the problem that the nail is colored by the incorporation of silver ion.

In the above, silver ion (Ag⁺) is used as an antibacterial material. Alternatively, another antibacterial material may be incorporated into the nail covering agent 110 (or the cover film 115) under the condition of providing an antibacterial effect. For example, titanium oxide may be incorporated instead of (or in addition to) silver ion. Still alternatively, silver ion, titanium oxide, and another antibacterial material may be used. Examples of the another antibacterial material include inorganic antibacterial agents (using the bacteriostatic function of metal ion), and organic antibacterial agents (antibacterial agents using organic substances; synthetic or natural antibacterial agents).

In addition, a far-infrared radiation material for emitting far-infrared rays may be incorporated into the nail covering agent 110. Examples of the far-infrared radiation material include ceramics and charcoal (bincho charcoal, etc.). By incorporating the far-infrared radiation material into the nail covering agent 110, the cover film 115 formed of the nail covering agent 110 is caused to emit far-infrared rays. Thus, an effect of promoting blood circulation by the far-infrared rays can be provided. According to the structure of this embodiment, the far-infrared radiation material may be incorporated at a content of about 10% (% by mass) (e.g., 5% to 20%) in the cover film 115.

As an example of main component of the cover film 115 in this embodiment, shellac is described. The present invention is not limited to this. Shellac is harmless to the human body and thus is preferable. In addition to shellac, another coating material (e.g., protein material such as collagen, gelatin or the like) may be incorporated. As a coating material incorporated together with shellac, a material which is as harmless to the human body as possible, like shellac, is preferable. Instead of shellac, hydroxypropylcellulose is usable. Hydroxypropylcellulose is a nonionic cellulose ether obtained by treating cellulose with sodium hydroxide and reacting the resultant substance with an etherizing agent such as propylene oxide or the like. Hydroxypropylcellulose is harmless to the human body and can form a transparent cover film.

In the above, the cover film 115 formed of the nail covering agent 110 is used as the base coat (and/or the top coat) for the nail seal 60. The cover film 115 in this embodiment is not limited to being used for the nail seal 60. For example, the cover film 115 formed of the nail covering agent 110 may be used as a base coat (and/or a top coat) for a fake nail (a type of artificial nail). The cover film 115 formed of the nail covering agent 110 may also be used as a base coat (and/or a top coat) for a gel nail containing an ultraviolet-curable resin as a coating material. In this embodiment, the term “fake nail” refers to a small piece having a curved surface which is obtained by industrially molding, for example, an acrylic resin to have a shape of a nail. A gel used to form the gel nail is a gel-like material containing, for example, a urethane-acrylic resin or the like as a main component. The gel is curable when being irradiated with light of an ultraviolet lamp.

In general, a gel nail is considered to be removable with acetone or the like. In actuality, however, a part of the cured ultraviolet-curable resin remains on the human nail, and occasionally, this ultraviolet-curable resin is physically scraped together with a part of the human nail by use of a file, a spatula or the like in addition to a chemical substance (acetone or the like). Such scraping damages the human nail, needless to say. In the case where the cover film 115 in this embodiment is used as the base coat for the gel nail, an effect that the gel nail can be removed without scraping, which would damage the human nail, is provided. Some gel nails have colors thereof changed after being cured with ultraviolet rays. In the case where the cover film 115 in this embodiment is used as the top coat, the influence of such a change of color can be alleviated or the changed color can be hidden with another color.

As a result of the studies made by the present inventor, the following was observed: when the cover film 115 (base coat) in this embodiment is formed before the gel nail is formed, the formed gel nail is less likely to be lifted, namely, is less likely to float to be delaminated. Usually, in order to prevent the gel nail from lifting, the human nail is filed (sanding) so that the adhesiveness with the gel nail is increased. However, sanding damages the human nail and also requires a troublesome work. According to the technique in this embodiment, the surface of the cover film 115 is almost ideally smooth from the viewpoint of adhesiveness. When a glass member and another glass member are put into contact with each other, a vacuum state is generated and the adhesiveness is strengthened. It is presumed for the same reason as for this phenomenon, the adhesiveness between the cover film 115 and the gel nail is improved.

When the gel nail is formed on the cover film 115 (base coat) in this embodiment, the gel nail and the cover film 115 are unlikely to have a gap therebetween. Since the gap is small, the adhesiveness is improved as described above, and also propagation of germs in the gap can be suppressed, which is highly hygienic. Usually, the gel nail tends to make the human nail cloudy. According to the technique in this embodiment, the cover film 115 (base coat) is less likely to make the human nail cloudy and also protects the human nail. When removing the gel nail, the cover film 115 (base coat) can protect the human nail. The cover film 115 provides another effect that the gel nail is more easily removed than the case where the cover film 115 is not provided.

An ultraviolet-curable resin may be incorporated into the nail covering agent 110 in this embodiment, so that the nail covering agent 110 is formed into a gel for the gel nail. In the case where a coating layer of the ultraviolet-curable resin for forming a gel nail does not contain shellac, the nail (human nail) is covered with the ultraviolet-curable resin and cannot breathe. When the coating layer contains shellac, there is an effect that the nail (human nail) can breathe because the coating layer of shellac allows air to permeate more than the coating layer of the ultraviolet-curable resin.

In the case where the nail covering agent 110 contains an antibacterial material, even a fake nail or a gel nail which is attached can be provided with an antibacterial characteristic by the cover film 115, which is highly advantageous hygienically. In addition, the cover film 115 formed of the nail covering agent 110 in this embodiment is usable as a base coat (and/or a top coat) for manicure or pedicure. When the nail covering agent 110 contains an antibacterial material, the manicure or pedicure can also be provided with an antibacterial characteristic by the cover film 115. In the case where the ultraviolet-curable resin for forming the gel nail contains an antibacterial material (e.g., silver ion), the gel nail itself can exert an antibacterial characteristic.

According to the structure of this embodiment, the nail covering agent 110 is mainly applied to the nail 52 of a hand. The present invention is not limited to this, and the nail covering agent 110 is usable to form the cover film 115 for covering the nails of a foot. The nail covering agent 110 is usable to form the cover film 115 for covering an artificial nail (fake nail, etc.) in addition to the human nail. In the case where the cover film 115 is formed and the nail seal 60 is pasted thereto, there is an effect that the nail seal 60 can be removed easily. Thus, it is possible to produce a unique artificial nail (fake nail) by pasting the nail seal 60 freely, and then to attach the artificial nail to the human nail.

As a result of studies made by the present inventor, the following knowledge was obtained: when pearl particles or mother-of-pearl particles are incorporated into the nail covering agent 110 containing shellac in this embodiment, the pearl color shines well. As described above, a manicure solution containing minute powder of shell or minute pulverized particles of pearl is disclosed in, for example, Japanese Patent Laid-Open Publication No. 2010-195689. In actuality, however, the pearl color in this publication does not shine well, and a manicure solution containing such minute powder of shell is not very popular. By contrast, when shellac-containing nail covering agent 110 in this embodiment which also contains mother-of-pearl (pearl oyster) particles (minute powder) is applied, a beautiful shine can be presented.

As described above, particles (minute powder) of mother-of-pearl (e.g., pearl oyster) can be produced by, for example, pulverizing mother-of-pearl. The mother-of-pearl particles have an average particle diameter of 1 μm to 10 μm and may be incorporated into the coating layer (cover film 115) of the nail covering agent 110 at a content of about 10% (% by mass) (e.g., 5% to 20%), although the above-mentioned particle diameter is merely exemplary.

The nail covering agent 110 in this embodiment may be formed of shellac, a solvent solution (e.g., ethanol), mother-of-pearl (or pearl) particles, and an ultraviolet-curable resin to form a gel for a gel nail. An antibacterial material such as silver ion or the like may be incorporated thereto. A gel for a gel nail containing mother-of-pearl (or pearl) particles and an ultraviolet-curable resin exhibits an antibacterial characteristic and also has been confirmed to have an effect of reducing the amount of a chemical substance (volatile organic compound). Owing to this, an antibacterial characteristic is provided and/or an uncomfortable odor caused by a volatile organic compound can be reduced.

The present inventor performed an antibacterial test on the cover film 115 (nail covering agent 110) in this embodiment. The cover film 115 was recognized to have an antibacterial characteristic. The nail covering agent 110 in this test example is formed of shellac, ethanol and silver ion. By volatilizing ethanol, the cover film 115 containing silver ion was produced. The content (% by mass) of silver ion was 5% or less, specifically, 2 to 3%. The test was performed by a method conformed to JIS Z 2801²⁰¹⁰, 5. As the bacteria, Staphylococcus aureus NBRC 12732 and Escherichia coli NBRC 3972 were supplied. The cover film 115 in this embodiment was recognized to be antibacterial against both of Staphylococcus aureus and Escherichia coli. As a non-processed test piece, a polyethylene film was used.

Regarding Staphylococcus aureus, in the case of the cover film 115 in this embodiment, the common logarithm value of live bacteria (after the bacteria were cultured for 24 hours) was <−0.20, and the antibacterial activity value was >4.9. Thus, the cover film 115 was recognized to be antibacterial against Staphylococcus aureus. In the case of the non-processed test piece provided as a reference example, the common logarithm value of live bacteria was 4.2 immediately after inoculation of a bacterium solution and 4.7 after the bacteria were cultivated for 24 hours. The non-processed test piece was not recognized to be antibacterial against Staphylococcus aureus. Regarding Escherichia coli, in the case of the cover film 115 in this embodiment, the common logarithm value of live bacteria was 2.6 (after the bacteria were cultured for 24 hours), and the antibacterial activity value was 3.1. Thus, the cover film 115 was recognized to be antibacterial against Escherichia coli. In the case of the non-processed test piece provided as a reference example, the common logarithm value of live bacteria was 4.2 immediately after inoculation of a bacterium solution and 5.8 after the bacteria were cultivated for 24 hours. The non-processed test piece was not recognized to be antibacterial against Escherichia coli. As the reference for approving that a test sample is antibacterial, an antibacterial activity value (bacteriostatic activity value) of 2.1 or greater can be used.

The present invention has been described by way of preferable embodiments, but the above description does not limit the present invention. The present invention may be modified in various manners.

### DESCRIPTION OF THE REFERENCE NUMERALS

10 Artificial nail composition
15 Cover film
20 Vessel main body
24 Bottle opening section
27 Pearl
30 Application section
32 Support rod
35 Lid section
40 Vessel main body
42 Application section
43 Conical section
45 Cap section
47 Sealing section
52 Nail
60 Nail seal
100 Vessel
110 Nail covering agent
115 Cover film
200 Vessel

What is claimed is:

1. An artificial nail composition for forming an artificial nail, the artificial nail composition being curable with ultraviolet rays, the artificial nail composition comprising:
an ultraviolet-curable resin; and
pearl powder dispersed in the ultraviolet-curable resin.

2. The artificial nail composition of claim 1, wherein the pearl powder is at least one selected from the group consisting of powder obtained as a result of pulverizing pearl and powder obtained as a result of pulverizing mother-of-pearl.

3. The artificial nail composition of claim 1, wherein the ultraviolet-curable resin has silver ion incorporated thereinto.

4. The artificial nail composition of claim 2, wherein the silver ion is contained at 10% by mass or less with respect to the entirety of the artificial nail composition.

5. The artificial nail composition of claim 1, wherein the ultraviolet-curable resin has at least one of shellac and hydroxypropylcellulose incorporated thereinto.

6. The artificial nail composition of claim 1, which contains at least one selected from the group consisting of titanium oxide powder and power stone powder.

7. The artificial nail composition of claim 1, wherein:
the artificial nail composition is accommodated in a vessel;
the vessel includes a vessel main body which is bottle-shaped;
the vessel main body is formed of a plastic material;
a bottle opening section having an opening through which the artificial nail composition can be introduced is provided in an upper part of the vessel main body;
a lid section for closing the opening is attached to the bottle opening section; and
an application section for applying the artificial nail composition is attached to the lid section.

8. The artificial nail composition of claim 1, wherein:
the artificial nail composition is accommodated in a vessel;
the vessel is pen-shaped;
the vessel includes a cylindrical vessel main body; and
the application section for applying the artificial nail composition is attached to a tip part of a conical section extending from the vessel main body.

9. A method for forming an artificial nail on a nail; the method comprising the steps of:
applying an artificial nail composition containing an ultraviolet-curable resin and pearl powder dispersed in the ultraviolet-curable resin on a surface of the nail; and
curing the artificial nail composition.

10. The method for forming an artificial nail of claim 9, wherein:
before the step of applying the artificial nail composition on the surface of the nail, a base coat containing at least one of shellac and hydroxypropylcellulose is formed as a base layer for the artificial nail composition.

11. The method for forming an artificial nail of claim 10, wherein the base coat contains silver ion.

12. The method for forming an artificial nail of claim 9, wherein:
after the step of curing the artificial nail composition, a top coat containing at least one of shellac and hydroxypropylcellulose is formed on a surface of the artificial nail composition.

13. The method for forming an artificial nail of claim 12, wherein the top coat contains silver ion.

14. A nail covering agent for coating a nail, the nail covering agent comprising:
a coating solution containing at least one of shellac and hydroxypropylcellulose; and
silver ion contained in the coating solution.

15. The nail covering agent of claim 14, wherein:
the coating solution includes:
the shellac; and
a solvent solution for dissolving the shellac;
the solvent solution is ethanol; and
the solvent solution does not contain any organic solvent other than ethanol.

16. The nail covering agent of claim 14, wherein:
the nail covering agent is accommodated in a vessel;
the vessel includes a vessel main body which is bottle-shaped;
the vessel main body is formed of a plastic material;
a bottle opening section having an opening through which the nail covering agent can be introduced is provided in an upper part of the vessel main body;
a lid section for closing the opening is attached to the bottle opening section; and
an application section for applying the nail covering agent is attached to the lid section.

17. The nail covering agent of claim 14, wherein:
the nail covering agent is accommodated in a vessel;
the vessel is pen-shaped;
the vessel includes a cylindrical vessel main body; and
the application section for applying the nail covering agent is attached to a tip part of a conical section extending from the vessel main body.

18. A method for forming a cover film on a nail, the method comprising the steps of:
applying a coating solution containing at least one of shellac and hydroxypropylcellulose and also silver ion on a surface of the nail; and
drying the coating solution.

19. The method for forming a cover film of claim 18, wherein:
after the step of drying the coating solution, a nail seal is pasted on a surface of the cover film formed of the coating solution.

20. The method for forming a cover film of claim 18, wherein:
after the step of drying the coating solution, a gel nail curable with ultraviolet rays is formed on a surface of the cover film formed of the coating solution.

21. The method for forming a cover film of claim 18, wherein:
in the step of applying the coating solution on the surface of the nail, the coating solution is applied to a surface of an artificial nail.

22. A nail covering agent for forming a cover film for coating a nail, the nail covering agent comprising:
a resin material for forming the cover film; and
an antibacterial agent contained in the resin material.

23. The nail covering agent of claim 22, wherein the cover film is one selected from the group consisting of a gel nail, a base coat and a top coat.
